(19) United States
(12) Patent Application Publication
(10) Pub. No.: US 2002/0142970 A1
(43) Pub. Date: Oct. 3, 2002

(54) Method for treating or preventing cardiovascular disease via administration of an ace inhibitor

(76) Inventor: Douglas E. Vaughan, Nashville, TN (US)

Correspondence Address: JENKINS & WILSON, PA, 3100 TOWER BLVD, SUITE 1400, DURHAM, NC, 27707, US

(21) Appl. No.: 09/974,703

(22) Filed: Oct. 10, 2001

Related U.S. Application Data
(60) Provisional application No. 60/239,324, filed on Oct. 10, 2000.

(51) Int. Cl. A61K 38/05; A61K 31/403

(52) U.S. Cl. 514/19; 514/412

(57) ABSTRACT

A method for preventing or reducing a risk of cardiovascular disease in a healthy subject via administration of an ACE inhibitor. Also provided is a method for reducing a plasma level of PAI-1 in a healthy subject via administration of an ACE inhibitor. The methods can be combined with hormone replacement therapy for improved effectiveness. Representative healthy subjects include post-menopausal women.

### CROSS REFERENCE TO RELATED APPLICATIONS

This application is based on and claims priority to U.S. Provisional Application Serial No. 60/239,324, filed Oct. 10, 2000, the entire contents of which are herein incorporated by reference.

### TECHNICAL FIELD

The present invention relates generally to therapeutic methods for cardiovascular disease. More particularly, the present invention relates to the modulation of plasminogen activator inhibitor—type 1 (PAI-1) levels via administration of an angiotensin converting enzyme (ACE) inhibitor to a subject in need thereof. Preferred subjects comprise post-menopausal women.

### TABLE OF ABBREVIATIONS

4G—PAI-1 polymorphism

5G—PAI-1 polymorphism

ACE—angiotensin converting enzyme

ACEI—ACE inhibition

HRT—hormone replacement therapy

PAI-1—plasminogen activator inhibitor type 1

tPA—tissue type plasminogen activator

### BACKGROUND OF THE INVENTION

Cardiovascular disease is the most common cause of death and disability in the United States. While the incidence of cardiovascular disease, and in particular coronary artery disease and acute myocardial infarction, is reduced in pre-menopausal women compared to that of age marked men, the incidence of cardiovascular disease increases rapidly in women following menopause. See Mendelsohn & Karas (1999) N Engl J Med 340:1801-1811.

Over the last ten years, a variety of new risk factors have been identified that can contribute to the development of heart ischemia. One factor that can contribute to increased risk of myocardial infarction is the circulating plasma level of plasminogen activator inhibitor-type 1(PAI-1) (Kohler & Grant, 2000). This factor is an inhibitor of the endogenous clot dissolving system in the body and elevated levels of PAI-1 appear to be an independent risk factor for acute myocardial infarction both in men and in women (Hamsten, 1993; Lijnen & Collen, 1996; Thogersen et al., 1998).

Some studies suggest that hormone replacement therapy (HRT) might provide an effective way of reducing the risk of heart disease in post-menopausal women. In particular, it has been shown that hormone replacement therapy with estrogen (such as that available under the registered trademark PREMARIN® estrogen from Wyeth-Ayerst Laboratories of St. Davids, Pa., U.S.A.) or estrogen plus progesterone can reduce plasma levels of PAI-1 (Koh et al., 1997). However, two recent prospective studies suggest that hormone replacement therapy might not be as effective as anticipated. See Herrington et al. (2000) N Engl J Med 343:522-529 and Hulley et al. (1998) JAMA 280:605-613. In addition, post-menopausal women who initiate HRT following a recent myocardial infarction show an increased risk of death (Alexander et al., 2001).

Thus, there exists a long-felt need in the art for prophylactic treatments to reduce the incidence of cardiovascular disease. To meet this need, the present invention provides a method for preventing or reducing a risk of cardiovascular disease in a healthy subject via administration of an ACE inhibitor, optionally in combination with hormone replacement therapy. The methods are preferably employed in the treatment of healthy subjects, particularly those subjects without a history of cardiovascular disease and/or subjects comprising post-menopausal women.

### SUMMARY OF THE INVENTION

The methods of the present invention are useful for treating a healthy subject, to thereby prevent or reduce a risk of cardiovascular disease or to reduce levels of PAI-1. The treatment comprises administration of an ACE inhibitor, optionally in combination with hormone replacement therapy.

A healthy subject can comprise a subject without prior incidence of cardiovascular disease, such as hypertension, congestive heart failure, left ventricular dysfunction, and prior myocardial infarct, or a subject free of induced activation of the renin-angiotensin system. For example, a healthy subject can comprise a post-menopausal female human subject. A healthy subject can also comprise a subject comprising a PAI-1 polymorphism, including a 4G PAI-1 polymorphism, wherein the PAI-1 polymorphism is correlated with an elevated level of PAI-1 when compared to a control level of PAI-1.

The present invention provides a method for preventing or significantly reducing a risk of cardiovascular disease in a healthy subject comprising administering an effective dose of an ACE inhibitor to the healthy subject, whereby the risk of cardiovascular disease in the healthy subject is prevented or significantly reduced.

In a preferred embodiment of the invention, risk for cardiovascular disease is determined by detecting an elevated level of PAI-1, and the methods for preventing or reducing a risk of cardiovascular disease comprise administering an ACE inhibitor to a healthy subject, whereby the plasma levels of PAI-1 are significantly reduced.

Thus, the present invention also provides a method for reducing a plasma level of PAI-1 in a healthy subject comprising administering an effective dose of an ACE inhibitor to the subject, whereby the plasma level of PAI-1 in the subject is reduced.

Preferably, the reduced risk or the reduced level of PAI-1 comprises a reduction of a plasma level of PAI-1 in a subject by at least about 35% compared to a baseline plasma level of PAI-1.

In a preferred embodiment of the invention, the method can comprise administering an ACE inhibitor to a healthy subject, wherein the subject comprises a 4G PAI-1 polymorphism, whereby a plasma level of PAI-1 in the subject is reduced by at least about 35% compared to a baseline plasma level of PAI-1.

Optionally, the methods of the present invention can be combined with hormone replacement therapy. For example, also provided is a method for preventing or significantly reducing a risk of cardiovascular disease in a healthy subject comprising co-administering an effective dose of an ACE inhibitor and an effective dose of a hormone to the healthy subject, whereby the risk of cardiovascular disease in the healthy subject is prevented or significantly reduced. Also provided is a method for reducing a plasma level of PAI-1 in a healthy subject comprising co-administering an effective dose of an ACE inhibitor and an effective dose of hormone to the subject, whereby the plasma level of PAI-1 in the subject is reduced.

When administration of an ACE inhibitor is combined with hormone replacement therapy, reduction of a risk of cardiovascular disease or a plasma level of PAI-1 in a subject preferably comprises a reduction of a plasma level of PAI-1 in the subject that is greater than a reduction of plasma PAI-1 levels following either treatment alone. Thus, in response to combined ACE inhibition and hormone replacement therapy, a reduction of a plasma level of PAI-1 can comprise a reduction of at least about 50% when compared to a baseline level of PAI-1.

A preferred ACE inhibitor that can be used in accordance with the methods of the present invention is ramipril. A preferred composition for hormone replacement that can be used in accordance with the present invention comprises PREMARIN® estrogen (Wyeth-Ayerst Laboratories of St. Davids, Pa., U.S.A).

### DETAILED DESCRIPTION OF THE INVENTION

I. Definitions

While the following terms are believed to be well understood by one of ordinary skill in the art, the following definitions are set forth to facilitate explanation of the invention.

The term “risk” refers to a calculated probability of occurrence and is generally determined by consideration of one or more risk factors. An elevated level of PAI-1 predicts an increased incidence of cardiovascular disease in patients with a prior incidence of myocardial infarction as well as in healthy patients. See Thogersen et al. (1998) Circulation 98:2241-2247; Lijnen & Collen (1996) Circulation 94:2052-2054; Hamsten (1993) Thromb Res 70:1-38. Thus, an elevated level of PAI-1 comprises a risk for developing cardiovascular disease.

The terms “significantly elevated” or “significantly increased”, as used herein to refer to an altered level of a measurable quality, for example a concentration or mass of plasma PAI-1, refers to a quantified change in the measurable quality that is larger than the margin of error inherent in the measurement technique, preferably an increase by about 30% or greater relative to a control or baseline measurement, more preferably an increase about 50% or greater, and most preferably an increase by about 70% or greater. A significant increase that is predictive of a myocardial infarct comprises an increase of about 32% to about 73% (Thogersen et al., 1998).

The term “significantly reduced”, as used herein to refer to an altered level of a measurable quality, for example a level of plasma PAI-1, refers to a quantified change in the measurable quality that is larger than the margin of error inherent in the measurement technique, preferably a decrease by about 10% or greater relative to a control or baseline measurement, more preferably a decrease about 30% or greater, and most preferably a decrease by about 50% or greater. In accordance with the methods of the present invention, a baseline measurement of PAI-1 levels comprises a level of PAI-1 in a healthy subject prior to receiving a treatment comprising ACE inhibition, hormone replacement therapy, or a combination thereof, as disclosed herein.

In accordance with the present invention, a control level of plasma PAI-1 in a broad population of healthy human adults comprises about 10.0 ng/ml for women and about 11.4 ng/ml for men as described by Thogersen et al. (1998) Circulation 98:2241-2247. A control level of plasma PAI-1 in healthy post-menopausal women comprises about 12.5 ng/ml to about 14.5 ng/ml (Table 1), and can vary up to about 26.0 ng/ml depending on genotype (Table 2).

The term “cardiovascular disease” refers to any of the diseases, whether congenital or acquired, of the heart and blood vessels. Representative cardiovascular diseases include but are not limited to coronary artery disease, hypertension, left ventricular dysfunction, myocardial infarction, ischemic heart disease, and congestive heart failure. Preferably, the terms “cardiovascular disease” refers to cardiovascular diseases in which plasma PAI-1 levels play causative, aggravating, indicating and/or other roles.

The term “coronary artery disease” refers to any disease of arteries that supply blood to the heart and/or arteries that surround the heart. For example, the term “coronary artery disease” includes thrombosis or a blood clot in the coronary arteries.

The term “hypertension” generally refers to elevated blood pressure. Typically, a hypertensive adult subject has a systolic pressure of 140 mm Hg or higher and/or a diastolic pressure of 90 mm Hg or higher. A normotenisve subject typically has a systolic pressure of about 120 mm Hg and a diastolic pressure of about 70 to 80 mm Hg.

The term “left ventricular dysfunction” generally refers to abnormal activity of the left ventricle of the heart including but not limited to myocardial infarction, ventricular fibrillation, heart arrhythmia, heart failure, heart rupture, phlebothrombosis, pulmonary embolism, pericarditis, mitral incompetence, and septum perforation.

The term “myocardial infarction” generally refers to death of a segment of heart muscle following an interruption in blood supply.

The term “ischemic heart disease” refers to any condition, for example constriction or blockage of blood vessels, resulting in inadequate flow of oxygen to the heart.

The term “renin-angiotensin system” refers to an endocrine cascade that regulates blood volume, vascular tone, and fibrinolysis, among other functions. ACE is the final enzyme in the cascade and the key catalytic step in the production of the peptide hormone angiotensin II. ACE cleaves two amino acids from the inactive prohormone angiotensin I (Ang I) to form the biologically active octapeptide Ang II, a potent vasoconstrictor and inhibitor of fibrinolysis.

The phrase “induced activation of the renin-angiotensin system” refers to a level of stimulation of the renin-angiotensin system in a subject. Activation of the renin-angiotensin system can disrupt fibrinolytic balance by stimulating excess production of PAI-1, thereby increasing the risk of thrombotic events. Thus, a level of stimulation can be measured, for example, by determining a significantly elevated level of plasma PAI-1 when compared to a baseline level of plasma PAI-1. See Brown et al. (1999) Hypertension 34:285-290 and Brown & Vaughan (1998) Circulation 97:1411-1420.

The term “about”, as used herein when referring to a measurable value such as a level of plasma PAI-1 (ng/ml), a dose, a temporal interval, etc., is meant to encompass variations of ±10%, more preferably ±5%, even more preferably ±1%, and still more preferably ±0.1% from the specified amount, as such variations are appropriate to perform the disclosed method.

II. Modulation of PAI-1 Levels by ACE Inhibition

The present invention pertains to methods for treating or reducing the risk of coronary artery disease or other cardiovascular disease via the modulation of plasminogen activator inhibitor-type 1 (PAI-1) levels. As disclosed herein, administration of an angiotensin converting enzyme (ACE) inhibitor to a healthy subject can reduce plasma PAI-1 levels.

The term “healthy” is used herein to refer to a condition that is free of hypertension, congestive heart failure, left ventricular dysfunction, and prior myocardial infarct, or induced activation of the renin-angiotensin system. Preferably, the term “healthy” also excludes a condition of high risk for cardiovascular disease. For example, human subjects greater than 55 years of age with evidence of vascular disease or diabetes are considered to be at high risk of cardiovascular disease.

Although numerous therapeutic uses of ACE inhibitors have been described, potential prophylactic uses of ACE inhibition have remained inconclusive. At present, there is no FDA approved indication or other disclosure in the art of ACE inhibition as an agent for reducing PAI-1 levels, and thereby reducing risk of cardiovascular disease, in a healthy population. See Brown & Vaughan (1998) Circulation 97:1411-1420. In addition, recent studies have suggested that ACE inhibition has no significant effect on PAI-1 levels in healthy subjects. See Lottermoser et al. (1999) Eur J Med Res 4:31-34. Thus, the present disclosure is believed to provide a first identification of ACE inhibition to lower plasma PAI-1 levels in healthy subjects, and preferably in normotensive post-menopausal female human subjects.

In a preferred embodiment of the invention, administration of an ACE inhibitor can be used to prevent or to reduce the risk of cardiovascular disease in healthy post-menopausal human subjects, a population that shows an increased incidence of developing cardiovascular disease. A risk of cardiovascular disease, and a reduction of the same, can be measured by determining a presence or level of any one of a number of risk factors associated with cardiovascular disease. For example, administration of rimipril can be used to significantly reduce plasma PAI-1 levels in healthy post-menopausal women. A comparison of the effects of PREMARIN® estrogen tablets with those of ramipril are described in Example 1.

The present invention further provides a method for preventing or reducing a risk of cardiovascular disease by co-administering an ACE inhibitor and estrogen, or an estrogen conjugate, to reduce plasma levels of PAI-1. For treatment of healthy post-menopausal women, a combined treatment including ACE inhibition and hormone replacement can effectively reduce plasma PAI-1 levels to a greater extent than either treatment alone, as described in Example 1.

The present invention further discloses that ACE inhibition can significantly reduce plasma PAI-1 levels most effectively in those subjects having higher baseline levels of PAI-1, and thus those subjects that are particularly in need of such a treatment. This aspect of preventive treatment for reducing PAI-1 levels is observed for ACE inhibition treatment, but is not observed for PREMARIN® estrogen treatment alone, as described further herein below.

A polymorphism in the human PAI-1 promoter results in altered levels of PAI-1 transcription and levels of circulating PAI-1. The polymorphism comprises a tract of 4 or 5 guanine residues, and the alleles are referred to as the “4G” or “5G” polymorphism, respectively. The 4G/4G genotype is associated with elevated levels of PAI-1 and a stronger correlation with the incidence of myocardial infraction when compared to the 4G/5G and 5G/5G genotypes (Dawson et al., 1993; Eriksson et al., 1995; Ossei-Gerning et al., 1997; Example 2). Administration of an ACE inhibitor effectively reduces plasma levels of PAI-1 in subjects having the 4G or 5G allele, although the a higher efficacy is observed in subjects having a 4G allele, as described in Example 2.

In accordance with the methods of the present invention, any ACE inhibitor can be used to prevent or reduce the risk of cardiovascular disease, or to reduce plasma levels of PAI-1 in a subject. The term “ACE inhibitor” refers to any agent that inhibits ACE and thereby prevents the conversion of angiotensin I to angiotensin II. For example, an ACE inhibitor can comprise amino acids, peptides, di- and tri-peptides, peptide mimetics, small molecules, antibodies, and analogues and derivatives thereof.

Representative ACE inhibitors include but are not limited to acylmercapto and mercaptoalkanoyl prolines, and derivatives thereof, such as captopril (U.S. Pat. No. 4,105,776) and zofenopril (U.S. Pat. No. 4,316,906); carboxyalkyl dipeptides such as enalapril (U.S. Pat. No. 4,374,829), lisinopril (U.S. Pat. No. 4,374,829), quinapril (U.S. Pat. No. 4,344,949), and perindopril (U.S. Pat. No. 4,508,729); carboxyalkyl dipeptide mimics such as cilazapril (U.S. Pat. No. 4,512,924) and benazapril (U.S. Pat. No. 4,410,520); phosphinylalkanoyl prolines such as fosinopril (U.S. Pat. No. 4,337,201) and trandolopril; phosphonate substituted amino or imino acids such as ceranapril (U.S. Pat. No. 4,452,790); and phosphoamidates (U.S. Pat. No. 4,432,971).

Additional ACE inhibitors that can be used in accordance with the methods of the present invention include ceranapril, alacepril, delapril, pentopril, quinapril, perindopril, cilazapril, and benazapril. See also U.S. Pat. Nos. 6,086,919; 5,977,159; 5,861,434; 5,852,047; 5,686,451; 5,212,165; 5,190,970; 5,166,143; 5,157,025; 5,098,889; 5,093,129; 5,061,694; 5,049,553; 5,037,821; 5,032,578; 5,015,633; 4,977,145; 4,931,430; 4,912,096; 4,748,160; 4,585,785; 4,568,675; 4,461,896; 4,447,419; and 4,442,089.

The term “ACE inhibitor” also refers to ACE inhibitor analogues. Methods for preparing ACE inhibitor analogues, for example to improve compound stability and potency, are described in U.S. Pat. Nos. 4,692,459; 4,692,458; 4,681,886; 4,626,545; 4,154,960; 4,154,840; 4,129,571; 4,113,751; 4,053,651; 4,052,511; and 4,046,889.

In a preferred embodiment of the invention, an ACE inhibitor comprises rimipril, which is available under the registered trademark ALTACE® from Hoechst Aktiengesellschaft, Frankfurt, Fed. Rep. Germany. Ramipril's chemical name is (2S,3aS,6aS)-1[(S)-N-[(S)-1-Carboxy-3-phenylpropyl]alanyl]octahydrocyclopenta[b]pyrrole-2-carboxylic acid, 1-ethyl ester. See U.S. Pat. No. 4,587,258.

The term “hormone”, as used herein to describe a composition that can be co-administered with an ACE inhibitor, generally refers to a composition for hormone replacement therapy comprising estrogen, progestin, or a combination thereof. Methods for formulation and administration of a hormone replacement composition to a subject are known in the art. Representative methods are described in U.S. Pat. Nos. 6,056,972; 5,891,868; 5,719,136; 5,512,557; and 4,729,999, among other places.

The term “estrogen” is used herein to generally refers to a natural or synthetic compound having an activity of an endogenous estrogen hormone (e.g., estriol, estrone, and estradiol). Thus, the term “estrogen” also encompasses estrogen-related compounds. Representative estrogen and estrogen-related compounds include but are not limited to 17-β-estradiol, conjugated estrogens (e.g., estrone sulfate, equilin, and 17-α-dihydroequilin), esterified estrogens, estradiol, estradiol valerate, estriol, estrone, estrone sulfate, estropipate, ethinyl estradiol, mestranol.

The term “progestin” generally refers to natural or synthetic compounds having an activity of an endogenous progesterone hormone. Thus the term “progestin” also encompasses progestin-related compounds. Representative progestin and progestin-related compounds include but are not limited to 17-deacetyl norgestimate, desogestrel, ethynodiol diacetate, levonorgestrel, medroxyprogesterone acetate, norethindrone, norethindrone acetate, norgestimate, norgestrel, progesterone, 3-keto desogestrel, chlormadinone acetate, cyproterone acetate, dienogest, dydrogesterone, gestodene, lynestrenol, megestrol, norethisterone, norethisterone acetate, norgestrienone, and quingestanol acetate.

III. Therapeutic Methods

III.A. Subiects

The methods of the present invention can be useful for treatment of a healthy subject, as defined herein above. The subject treated in the present invention in its many embodiments is desirably a human subject, although it is to be understood that the principles of the invention indicate that the invention is effective with respect to all vertebrate species, including mammals, which are intended to be included in the term “subject”. In this context, a mammal is understood to include any mammalian species in which treatment is desirable, particularly agricultural and domestic mammalian species.

The term “subject” as used herein refers to any invertebrate or vertebrate species. The methods of the present invention are particularly useful in the treatment of warm-blooded vertebrates. Thus, the invention concerns mammals and birds. More particularly, contemplated is the treatment and/or diagnosis of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economical importance to humans. Thus, contemplated is the treatment of livestock, including, but not limited to, domesticated swine (pigs and hogs), ruminants, horses, poultry, and the like.

III.B. Formulation

A therapeutic composition (e.g., a composition comprising an ACE inhibitor, a hormone or hormone conjugate, or a combination thereof) preferably comprises a composition that includes a pharmaceutically acceptable carrier. Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions used in the methods can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art. For example, an ACE inhibitor can be formulated in combination with hydrochlorothiazide, and as a pH stabilized core having an enteric or delayed release coating which protects the ACE inhibitor until it reaches the colon.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

III.C. Dose

The term “effective amount” is used herein to refer to an amount of the therapeutic composition (e.g., a composition comprising an ACE inhibitor, a hormone or hormone conjugate, or a combination thereof) sufficient to produce a measurable biological response (e.g., a reduction of plasma PAI-1 levels). Actual dosage levels of active ingredients in a therapeutic composition of the invention can be varied so as to administer an amount of the active compound(s) that is effective to achieve the desired therapeutic response for a particular subject and/or application. The selected dosage level will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art of medicine.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich et al., 1966). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al. (1966) Cancer Chemother Rep 50:219-244. Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

For oral administration, a satisfactory result can be obtained employing the ACE inhibitor in an amount ranging from about 0.01 mg/kg to about 100 mg/kg and preferably from about 0.1 mg/kg to about 5 mg/kg. A preferred oral dosage form, such as tablets or capsules, will contain the ACE inhibitor in an amount ranging from about 0.1 to about 500 mg, preferably from about 2 to about 50 mg, and more preferably from about 10 to about 25 mg. Representative doses for oral administration are also provided in Examples 1 and 2.

For parenteral administration, the ACE inhibitor can be employed in an amount ranging from about 0.005 mg/kg to about 10 mg/kg and preferably from about 0.005 mg/kg to about 2 mg/kg.

ACE inhibitors are typically administered at a initial acceptable dose, and the dose is escalated up as the subject tolerates it. For example, for administration to a human adult, a test dose of enalapril is 5 mg/day, which is then increased up to 10-20 mg/day, once a day, as the patient tolerates it. As another example, captopril is initially administered orally to human patients in a test dose of 6.25 mg/day and the dose is then escalated, as the patient tolerates it, to 25 mg twice per day (BID) or three times per day (TID) and can be titrated to 50 mg BID or TID. Tolerance level is estimated by determining whether decrease in blood pressure is accompanied by signs of hypotension. If indicated, the dose can be increased up to 100 mg BID or TID.

For additional guidance regarding formulation and dose, see U.S. Pat. Nos. 5,326,902; 5,234,933; PCT International Publication No. WO 93/25521; Berkow et al. (1997) The Merck Manual of Medical Information, Home ed. Merck Research Laboratories, Whitehouse Station, N.J.; Goodman et al. (1996) Goodman & Gilman's the Pharmacological Basis of Therapeutics, 9th ed. McGraw-Hill Health Professions Division, New York; Ebadi (1998) CRC Desk Reference of Clinical Pharmacology. CRC Press, Boca Raton, Fla.; Katzung (2001) Basic & Clinical Pharmacology, 8th ed. Lange Medical Books/McGraw-Hill Medical Pub. Division, New York; Remington et al. (1975) Remington's Pharmaceutical Sciences, 15th ed. Mack Pub. Co., Easton, Pa.; and Speight et al. (1997) Avery's Drug Treatment: A Guide to the Properties, Choice, Therapeutic Use and Economic Value of Drugs in Disease Management, 4th ed. Adis International, Auckland/Philadelphia; Duch et al. (1998) Toxicol Lett 100-101:255-263.

III.D. Administration

Suitable methods for administering to a subject an ACE inhibitor, a hormone, hormone conjugate, or combination thereof in accordance with the methods of the present invention include but are not limited to systemic administration, parenteral administration (including intravascular, intramuscular, intraarterial administration), oral delivery, buccal delivery, subcutaneous administration, inhalation, intratracheal installation, surgical implantation, transdermal delivery, local injection, and hyper-velocity injection/bombardment. Where applicable, continuous infusion can enhance drug accumulation at a target site (e.g., U.S. Pat. No. 6,180,082).

The particular mode of drug administration used in accordance with the methods of the present invention depends on various factors, including but not limited to the vector and/or drug carrier employed, the severity of the condition to be treated, and mechanisms for metabolism or removal of the drug following administration.

### EXAMPLES

The following Examples have been included to illustrate preferred modes of the invention. Certain aspects of the following Examples are described in terms of techniques and procedures found or contemplated by the present inventor to work well in the practice of the invention. These Examples are exemplified through the use of standard laboratory practices of the inventor. In light of the present disclosure and the general level of skill in the art, those of skill will appreciate that the following examples are intended to be exemplary only and that numerous changes, modifications and alterations can be employed without departing from the spirit and scope of the invention.

### Example 1

PAI-1 is the major physiological inhibitor of plasminogen activation, and increased levels of plasma PAI-1 can contribute to the risk of cardiovascular disease. Plasma PAI-1 levels can be reduced by treatment with conjugated estrogen (in post-menopausal women) and by ACE inhibitors (in subjects having cardiovascular disease, in subjects following a myocardial infarct, and in subjects having an activated renin-angiotensin system). In this Example, healthy, normotensive post-menopausal women (n=14) were randomized in a single blind crossover trial to receive PREMARIN® estrogen (0.625 mg daily), ramipril (10 mg daily), or a combination of PREMARIN® estrogen and ramipril. Each treatment period lasted 4 weeks followed by a 4 week washout period.

Treatment with PREMARIN® estrogen produced a decrease in PAI-1 levels as known in the art. Surprisingly, ACE inhibition also effectively lowered PAI-1 production in healthy normotensive, post-menopausal subjects (Table 1). Subjects receiving PREMARIN® estrogen treatment for 4 weeks showed a 43% decrease in plasma PAI-1 levels (P<0.005). Subjects receiving ramipril therapy showed a 38% decrease in plasma PAI-1 levels (P<0.002). Subjects receiving a combination of PREMARIN® estrogen and ramipril treatment showed a 53% decrease in PAI-1 levels. In all subjects, a change in plasma tPA levels was not observed. Thus, PREMARIN® estrogen and ramipril treatments also improved the molar ratio of PAI-1/tPA in plasma.

TABLE 1
PAl-1 and tPA Levels Following HRT and ACEI

| Treatment | PAl-1 ng/ml | p value | tPA ng/ml |
|---|---|---|---|
| Baseline | 13.7 ± 10.1 | — | 5.6 ± 3.0 |
| PREMARIN ® | 7.8 ± 9.5 | 0.0023 | 6.2 ± 4.0 |
| estrogen | | | |
| Baseline | 12.5 ± 9.7 | — | 6.6 ± 3.2 |
| Ramipril | 7.9 ± 5.8 | 0.0013 | 6.5 ± 2.4 |
| Baseline | 14.4 ± 10.2 | — | 6.8 ± 3.0 |
| Combination | 8.8 ± 7.9 | 0.0091 | 5.7 ± 1.9 |

Baseline, plasma PAl-1 levels prior to treatment; SD, standard deviation

### Example 2

A combination of genetic and environmental factors regulates plasma PAI-1 levels in humans. In this Example, the effects of hormone replacement therapy (HRT) and ACE inhibition (ACEI) as a function of genotype on plasma PAI-1 levels in healthy post-menopausal women (n=14) were examined. Allelic status of the 4G/5G PAI-1 polymorphism influenced plasma PAI-1 levels and treatment efficacy (Table 2).

The 4G/5G polymorphism in the PAI-1 promoter is a determinant of plasma PAI-1 levels in post-menopausal women. Post-menopausal women having the 4G/4G PAI-1 polymorphism displayed the highest levels of plasma PAI-1 (25.7 ng/ml ±9.2), post-menopausal women having the 4G/5G PAI-1 polymorphism showed intermediate levels of plasma PAI-1 (11.7 ng/ml ±5.5), and post-menopausal women having the 5G/5G PAI-1 polymorphism showed lower levels of plasma PAI-1 (6.2 mg/ml ±4.3). The elevated levels of plasma PAI-1 in post-menopausal women having the 4G/4G PAI-1 polymorphism or the 4G/5G polymorphism suggest that they harbor a higher risk of cardiovascular disease.

HRT and ACE inhibition effectively lowered plasma levels of PAI-1 in all subjects. Overall HRT with PREMARIN® estrogen (0.625 mg/daily) reduced PAI-1 levels by average of 43% (P<0.005). The effect of HRT was highly dependent upon genotype, with PAI-1 levels falling 22% in 4G homozygotes and 72% in 5G homozygotes (P<0.02). In contrast, ACE inhibition with ramipril (10 mg/daily) lowered mean plasma PAI-1 to a similar extent (38% P<0.002), but reduced levels most effectively in subjects with the 4G allele (4G/4G 39%, 4G/5G 44%, 5G/5G 13%). Thus, ACE inhibition provides the most effective treatment for lowering the risk of cardiovascular disease in post-menopausal subjects showing the most need thereof.

TABLE 2
PAI-1 Plasma Levels (mean +/− SD mg/ml)

| Genotype | Baseline | HRT | p | Baseline | ACEI | p |
|---|---|---|---|---|---|---|
| 4G/4G | 25.7 ± 9.2 | 19.7 ± 10.3 | NS | 25.0 ± 11.1 | 16.2 ± 11.0 | <0.002 |
| 4G/5G | 11.7 ± 5.5 | 4.0 ± 3.2 | NS | 11.7 ± 5.51 | 5.2 ± 2.4 | <0.05 |
| 5G/5G | 6.2 ± 4.3 | 1.9 ± 2.1 | <0.02 | 6.1 ± 4.3 | 4.6 ± 1.6 | NS |

SD, standard deviation; Baseline, plasma levels prior to treatment; HRT, plasma PAI-1 levels following hormone replacement therapy; ACEI, plasma levels following ACE inhibition; NS, not significant.

### REFERENCES

The references listed below as well as all references cited in the specification are incorporated herein by reference to the extent that they supplement, explain, provide a background for or teach methodology, techniques and/or compositions employed herein.

Alexander K P, Newby L K, Hellkamp A S, Harrington R A, Peterson E D, Kopecky S, Langer A, O'Gara P, O'Connor C M, Daly R N, Califf R M, Khan S & Fuster V (2001) Initiation of Hormone Replacement Therapy after Acute Myocardial Infarction Is Associated with More Cardiac Events During Follow-Up. J Am Coll Cardiol38:1-7.

Berkow R, Beers M H & Fletcher A J (1997) The Merck Manual of Medical Information, Home ed. Merck Research Laboratories, Whitehouse Station, N.J.

Brown N J & Vaughan D E (1998) Angiotensin-Converting Enzyme Inhibitors. Circulation 97:1411-1420.

Brown N J, Agirbasli M & Vaughan D E (1999) Comparative Effect of Angiotensin-Converting Enzyme Inhibition and Angiotensin Ii Type 1 Receptor Antagonism on Plasma Fibrinolytic Balance in Humans. Hypertension 34:285-290.

Dawson S J, Wiman B, Hamsten A, Green F, Humphries S & Henney A M (1993) The Two Allele Sequences of a Common Polymorphism in the Promoter of the Plasminogen Activator Inhibitor-1 (Pai-1) Gene Respond Differently to lnterleukin-1 in Hepg2 Cells. J Biol Chem 268:10739-10745.

Duch D S, Rehberg B & Vysotskaya T N (1998) Volatile Anesthetics Significantly Suppress Central and Peripheral Mammalian Sodium Channels. Toxicol Lett 100-101:255-263.

Ebadi M S (1998) CRC Desk Reference of Clinical Pharmacology. CRC Press, Boca Raton, Fla.

Eriksson P, Kallin B, van 't Hooft F M, Bavenholm P & Hamsten A (1995) Allele-Specific Increase in Basal Transcription of the Plasminogen-Activator Inhibitor 1 Gene Is Associated with Myocardial Infarction. Proc Natl Acad Sci USA 92:1851-1855.

Freireich E J, Gehan E A, Rall D P, Schmidt L H & Skipper H E (1966) Quantitative Comparison of Toxicity of Anticancer Agents in Mouse, Rat, Hamster, Dog, Monkey, and Man. Cancer Chemother Rep 50:219-244.

Goodman L S, Gilman A, Hardman J G, Gilman A G & Limbird L E (1996) Goodman & Gilman's the Pharmacological Basis of Therapeutics, 9th ed. McGraw-Hill Health Professions Division, New York.

Hamsten A (1993) The Hemostatic System and Coronary Heart Disease. Thromb Res 70:1-38.

Herrington D M, Reboussin D M, Brosnihan K B, Sharp P C, Shumaker S A, Snyder T E, Furberg C D, Kowalchuk G J, Stuckey T D, Rogers W J, Givens D H & Waters D (2000) Effects of Estrogen Replacement on the Progression of Coronary-Artery Atherosclerosis. N Engl J Med 343:522-529.

Hulley S, Grady D, Bush T, Furberg C, Herrington D, Riggs B & Vittinghoff E (1998) Randomized Trial of Estrogen Plus Progestin for Secondary Prevention of Coronary Heart Disease in Post-menopausal Women. Heart and Estrogen/Progestin Replacement Study (Hers) Research Group. JAMA 280:605-613.

Katzung B G (2001) Basic & Clinical Pharmacology, 8th ed. Lange Medical Books/McGraw-Hill Medical Pub. Division, New York.

Koh K K, Mincemoyer R, Bui M N, Csako G, Pucino F, Guetta V, Waclawiw M & Cannon R O, 3rd (1997) Effects of Hormone-Replacement Therapy on Fibrinolysis in Post-menopausal Women. N Engl J Med 336:683-690.

Kohler H P & Grant P J (2000) Plasminogen-Activator Inhibitor Type 1 and Coronary Artery Disease. N Engl J Med 342:1792-1801.

Lijnen H R & Collen D (1996) Impaired Fibrinolysis and the Risk for Coronary Heart Disease. Circulation 94:2052-2054.

Lottermoser K, Wostmann B, Weisser B, Hertfelder H J, Schmitz U, Vetter H & Dusing R (1999) Effects of Captopril on Fibrinolytic Function in Healthy Humans. Eur J Med Res 4:31-34.

Mendelsohn M E & Karas R H (1999) The Protective Effects of Estrogen on the Cardiovascular System. N Engl J Med 340:1801-1811.

Ossei-Gerning N, Mansfield M W, Stickland M H, Wilson I J & Grant P J (1997) Plasminogen Activator Inhibitor-1 Promoter 4g/5g Genotype and Plasma Levels in Relation to a History of Myocardial Infarction in Patients Characterized by Coronary Angiography. Arterioscler Thromb Vasc Biol 17:33-37.

Remington J P, Osol A, Anderson J T, Hoover J E & Skolaut M W (1975) Reminqton's Pharmaceutical Sciences, 15th ed. Mack Pub. Co., Easton, Pa.

Speight T M, Holford N H G & Avery G S (1997) Avery's Drug Treatment: A Guide to the Properties, Choice, Therapeutic Use and Economic Value of Drugs in Disease Management, 4th ed. Adis International, Auckland/Philadelphia, Pa.

Thogersen A M, Jansson J H, Boman K, Nilsson T K, Weinehall L, Huhtasaari F & Hallmans G (1998) High Plasminogen Activator Inhibitor and Tissue Plasminogen Activator Levels in Plasma Precede a First Acute Myocardial Infarction in Both Men and Women: Evidence for the Fibrinolytic System as an Independent Primary Risk Factor. Circulation 98:2241-2247.

U.S. Pat. No. 4,046,889

U.S. Pat. No. 4,052,511

U.S. Pat. No. 4,053,651

U.S. Pat. No. 4,105,776

U.S. Pat. No. 4,113,751

U.S. Pat. No. 4,129,571

U.S. Pat. No. 4,154,840

U.S. Pat. No. 4,154,960

U.S. Pat. No. 4,316,906

U.S. Pat. No. 4,337,201

U.S. Pat. No. 4,344,949

U.S. Pat. No. 4,374,829

U.S. Pat. No. 4,410,520

U.S. Pat. No. 4,337,201

U.S. Pat. No. 4,442,089

U.S. Pat. No. 4,447,419

U.S. Pat. No. 4,461,896

U.S. Pat. No. 4,508,729

U.S. Pat. No. 4,512,924

U.S. Pat. No. 4,452,790

U.S. Pat. No. 4,568,675

U.S. Pat. No. 4,585,758

U.S. Pat. No. 4,587,258

U.S. Pat. No. 4,626,545

U.S. Pat. No. 4,681,886

U.S. Pat. No. 4,692,458

U.S. Pat. No. 4,692,459

U.S. Pat. No. 4,729,999

U.S. Pat. No. 4,748,160

U.S. Pat. No. 4,912,096

U.S. Pat. No. 4,931,430

U.S. Pat. No. 4,977,145

U.S. Pat. No. 5,015,633

U.S. Pat. No. 5,032,578

U.S. Pat. No. 5,037,821

U.S. Pat. No. 5,049,553

U.S. Pat. No. 5,061,694

U.S. Pat. No. 5,093,129

U.S. Pat. No. 5,098,889

U.S. Pat. No. 5,157,025

U.S. Pat. No. 5,166,143

U.S. Pat. No. 5,190,970

U.S. Pat. No. 5,212,165

U.S. Pat. No. 5,234,933

U.S. Pat. No. 5,326,902

U.S. Pat. No. 5,512,557

U.S. Pat. No. 5,686,451

U.S. Pat. No. 5,719,136

U.S. Pat. No. 5,852,047

U.S. Pat. No. 5,861,434

U.S. Pat. No. 5,891,868

U.S. Pat. No. 5,977,159

U.S. Pat. No. 6,056,972

U.S. Pat. No. 6,086,919

U.S. Pat. No. 6,180,082

WO 93/25521

It will be understood that various details of the invention may be changed without departing from the scope of the invention. Furthermore, the foregoing description is for the purpose of illustration only, and not for the purpose of limitation—the invention being defined by the claims.

What is claimed is:

1. A method for preventing or significantly reducing a risk of cardiovascular disease in a healthy subject comprising administering an effective dose of an ACE inhibitor to the healthy subject, whereby the risk of cardiovascular disease in the healthy subject is prevented or significantly reduced.

2. The method of claim 1, wherein the ACE inhibitor comprises ramipril.

3. The method of claim 1, wherein the healthy subject comprises a subject free of hypertension, congestive heart failure, left ventricular dysfunction, prior myocardial infarct, and induced activation of the renin-angiotensin system.

4. The method of claim 1, wherein the healthy subject comprises a post-menopausal female human subject.

5. The method of claim 1, wherein the healthy subject comprises a subject comprising a PAI-1 polymorphism, wherein the PAI-1 polymorphism results in an elevated level of PAI-1 when compared to a control level of PAI-1.

6. The method of claim 5, wherein the PAI-1 polymorphism comprises a 4G PAI-1 polymorphism.

7. The method of claim 1, wherein reducing the risk of cardiovascular disease in the healthy subject comprises significantly reducing a plasma level of PAI-1 in the healthy subject.

8. The method of claim 1, wherein the significantly reducing a plasma level of PAI-1 comprises reducing a plasma level of PAI-1 by at least about 35% compared to a baseline plasma level of PAI-1.

9. The method of claim 8, wherein the healthy subject comprises a subject comprising a PAI-1 polymorphism, wherein the PAI-1 polymorphism is correlated with elevated levels of PAI-1 activity, and wherein the significantly reducing a plasma level of PAI-1 comprises reducing a plasma level of PAI-1 by at least about 35% compared to a baseline plasma level of PAI-1.

10. A method for reducing a plasma level of PAI-1 in a healthy subject comprising administering an effective dose of an ACE inhibitor to the subject, whereby the plasma level of PAI-1 in the subject is reduced.

11. The method of claim 10, wherein the ACE inhibitor comprises ramipril.

12. The method of claim 10, wherein the healthy subject comprises a subject free of hypertension, congestive heart failure, left ventricular dysfunction, and prior myocardial infarct, and induced activation of the renin-angiotensin system.

13. The method of claim 10, wherein the healthy subject comprises a post-menopausal female human subject.

14. The method of claim 10, wherein the healthy subject comprises a subject comprising a PAI-1 polymorphism, wherein the PAI-1 polymorphism is correlated with an elevated level of PAI-1 when compared to a control level of PAI-1.

15. The method of claim 14, wherein the PAI-1 polymorphism comprises a 4G PAI-1 polymorphism.

16. The method of claim 10, wherein the significantly reducing a plasma level of PAI-1 comprises reducing a plasma level of PAI-1 by at least about 35% compared to a baseline plasma level of PAI-1.

17. The method of claim 16, wherein the healthy subject comprises a subject comprising a PAI-1 polymorphism, wherein the PAI-1 polymorphism is correlated with elevated levels of PAI-1 activity, and wherein the significantly reducing a plasma level of PAI-1 comprises reducing a plasma level of PAI-1 by at least about 35% compared to a baseline plasma level of PAI-1.

18. A method for preventing or significantly reducing a risk of cardiovascular disease in a healthy subject comprising co-administering an effective dose of an ACE inhibitor and an effective dose of hormone, or a conjugate thereof, to the healthy subject, whereby the risk of cardiovascular disease in the healthy subject is prevented or significantly reduced.

19. The method of claim 18, wherein the ACE inhibitor comprises ramipril.

20. The method of claim 19, wherein the hormone comprises PREMARIN® estrogen.

21. The method of claim 18, wherein the healthy subject comprises a subject free of hypertension, congestive heart failure, left ventricular dysfunction, prior myocardial infarct, and induced activation of the renin-angiotensin system.

22. The method of claim 18, wherein the healthy subject comprises a post-menopausal female human subject.

23. The method of claim 18, wherein the healthy subject comprises a subject comprising a PAI-1 polymorphism, wherein the PAI-1 polymorphism is correlated with an elevated level of PAI-1 when compared to a control level of PAI-1.

24. The method of claim 23, wherein the PAI-1 polymorphism comprises a 4G PAI-1 polymorphism.

25. The method of claim 18, wherein reducing the risk of cardiovascular disease in the healthy subject comprises significantly reducing a plasma level of PAI-1 in the healthy subject.

26. The method of claim 25, wherein the significantly reducing a plasma level of PAI-1 in the healthy subject comprises reducing the plasma level of PAI-1, wherein the reducing is to a greater extent when compared to a reducing a plasma of PAI-1 following administration of an ACE inhibitor or hormone to the subject.

27. The method of claim 25, wherein the significantly reducing a plasma level of PAI-1 comprises reducing a plasma level of PAI-1 by at least about 50% when compared to a baseline plasma level of PAI-1.

28. A method for reducing a plasma level of PAI-1 in a healthy subject comprising co-administering an effective dose of an ACE inhibitor and an effective dose of hormone to the subject, whereby the plasma level of PAI-1 in the subject is reduced.

29. The method of claim 28, wherein the ACE inhibitor comprises ramipril.

30. The method of claim 28, wherein the hormone comprises PREMARIN® estrogen.

31. The method of claim 28, wherein the healthy subject comprises a subject free of hypertension, congestive heart failure, left ventricular dysfunction, prior myocardial infarct, and induced activation of the renin-angiotensin system.

32. The method of claim 28, wherein the healthy subject comprises a post-menopausal female human subject.

33. The method of claim 28, wherein the healthy subject comprises a subject comprising a PAI-1 polymorphism, wherein the PAI-1 polymorphism is correlated with an elevated level of PAI-1 when compared to a control level of PAI-1.

34. The method of claim 28, wherein the PAI-1 polymorphism comprises a 4G PAI-1 polymorphism.

35. The method of claim 28, wherein the significantly reducing a plasma level of PAI-1 in the healthy subject comprises reducing the plasma level of PAI-1, wherein the reducing is to a greater extent when compared to a reducing a plasma of PAI-1 following administration of an ACE inhibitor or estrogen to the subject.

36. The method of claim 28, wherein the significantly reducing a plasma level of PAI-1 comprises reducing a plasma level of PAI-1 by at least about 50% when compared to a baseline plasma level of PAI-1.
